(19) United States
(12) Patent Application Publication
(10) Pub. No.: US 2023/0190239 A1
(43) Pub. Date: Jun. 22, 2023

(54) ULTRASONIC DIAGNOSIS APPARATUS AND ULTRASOUND IMAGE TRANSFER METHOD

(71) Applicant: FUJIFILM Healthcare Corporation, Kashiwa-shi (JP)

(72) Inventor: Fumie Matsuda, Chiba (JP)

(21) Appl. No.: 17/970,693

(22) Filed: Oct. 21, 2022

(30) Foreign Application Priority Data
Dec. 20, 2021 (JP) 2021-206104

(51) Int. Cl. A61B 8/00; G01S 7/00; G16H 30/20

(52) U.S. Cl. CPC A61B 8/54; A61B 8/463; A61B 8/565; G01S 7/003; G16H 30/20

(57) ABSTRACT

Based on a set of preset data for setting operation conditions, which is selected prior to an ultrasonic examination, a transfer control unit determines an image attribute of an image obtained in the ultrasonic examination. The transfer control unit selects, from among a plurality of servers, a transfer destination server assigned a server attribute suitable for the image attribute. The transfer control unit transfers the image to the transfer destination server.

### CROSS REFERENCE TO RELATED APPLICATION

This application claims priority to Japanese Patent Application No. 2021-206104 filed on Dec. 20, 2021, which is incorporated herein by reference in its entirety including the specification, claims, drawings, and abstract.

### TECHNICAL FIELD

The present disclosure relates to an ultrasonic diagnosis apparatus and an ultrasound image transfer method, and more particularly to a technique of transferring ultrasound images from an ultrasonic diagnosis apparatus to servers.

### BACKGROUND

For example, in a large medical institution, an ultrasonic diagnosis apparatus is connected to a plurality of servers via a network. Each server is a file server (or an image storage) that stores and accumulates a large number of images obtained in ultrasonic examinations. Generally, each server is assigned a specific area of examination (which may also be referred to as a specific field of clinical practice or a specific body organ). In one example, images obtained in an ultrasonic examination of the abdomen are transferred from an ultrasonic diagnosis apparatus to a specific server for storing abdomen images.

Patent Document 1 (JP 2009-125385 A) shows, in FIG. 6, a screen for selecting a transfer destination server from among a plurality of servers. Selection of the transfer destination server is performed by a user. In Patent Document 1, a technique of automatically selecting a transfer destination server is nowhere described.

In transferring images from an ultrasonic diagnosis apparatus to a plurality of servers, it is necessary to select a transfer destination server for each image or for each group of images. If an examiner (such as a medical doctor or a medical technologist), who is the user, is requested to designate a transfer destination server at each time of transfer, there may occur the problems of increased workload for the examiner and incorrect selection of transfer destination servers. In particular, from the perspective of management and reuse of medical information, the latter problem is a serious problem that should be solved.

The present disclosure is directed to reducing the examiner's workload when images obtained in an ultrasonic examination are to be transferred to servers. Alternatively, the present disclosure is directed to correctly transferring images obtained in an ultrasonic examination to servers suitable for those images.

### SUMMARY

An ultrasonic diagnosis apparatus according to the present disclosure includes: a determining unit configured to determine, based on operation condition setting information selected prior to an ultrasonic examination, an image attribute of an image obtained in the ultrasonic examination; a selecting unit configured to select, from among a plurality of servers, a transfer destination server assigned a server attribute suitable for the image attribute; and a transfer unit configured to transfer the image to the transfer destination server.

An ultrasound image transfer method according to the present disclosure includes: determining, based on operation condition setting information selected prior to an ultrasonic examination, an image attribute of an image obtained in the ultrasonic examination; selecting, from among a plurality of servers, a transfer destination server assigned a server attribute suitable for the image attribute; and transferring the image to the transfer destination server.

### BRIEF DESCRIPTION OF DRAWINGS

Embodiment(s) of the present disclosure will be described based on the following figures, wherein:

FIG. 1 is a block diagram showing an ultrasonic diagnosis apparatus according to an embodiment;

FIG. 2 is a block diagram showing an example configuration of a transfer control unit;

FIG. 3 is a diagram showing a first example of a preset data management table;

FIG. 4 is a diagram showing a second example of the preset data management table;

FIG. 5 is a diagram showing an example of an image management table;

FIG. 6 is a diagram showing an example of a server management table;

FIG. 7 is a diagram illustrating a method of designating server attributes;

FIG. 8 is a flowchart illustrating an operation at the time of storing an image;

FIG. 9 is a flowchart illustrating an operation at the time of transferring an image;

FIG. 10 is a diagram showing another example of the image management table; and

FIG. 11 is a schematic diagram illustrating a variant example of the operation at the time of transferring an image.

### DESCRIPTION OF EMBODIMENTS

Embodiments will now be described by reference to the drawings.

### (1) Overview of Embodiments

An ultrasonic diagnosis apparatus according to an embodiment comprises a determining unit, a selecting unit, and a transfer unit. The determining unit determines, based on operation condition setting information selected prior to an ultrasonic examination, an image attribute of an image obtained in the ultrasonic examination. The selecting unit selects, from among a plurality of servers, a transfer destination server assigned a server attribute suitable for the image attribute. The transfer unit transfers the image to the transfer destination server.

According to the above-described configuration, a transfer destination server suitable for a transfer target image is selected automatically at the time of image transfer. The transfer target image is then transferred to that transfer destination server. Since it is unnecessary for the examiner to select a transfer destination at the time of image transfer, the examiner's workload can be reduced, and incorrect selection of a transfer destination server can be avoided.

When an ultrasonic examination is to be performed, prior to the ultrasonic examination, operation conditions of the ultrasonic diagnosis apparatus are set. More specifically, operation conditions appropriate for the area of examination (i.e., the field of clinical practice, the tissue to be examined, or the like) are set. With that as background, in the above-described configuration, an image attribute is determined based on operation condition setting information. The operation condition setting information includes preset data, operation conditions specifically set for the ultrasonic diagnosis apparatus, and the like.

In an ultrasonic diagnosis apparatus, a plurality of sets of preset data are generally provided. Each set of preset data includes parameters that define operation conditions of the ultrasonic diagnosis apparatus. A selected specific set of preset data is used as the operation condition setting information. The image attribute is determined based on that specific set of preset data.

Typically, one or a plurality of sets of preset data are provided for each individual area of examination. A set of preset data customized by an examiner may also be used. In any case, when an ultrasonic examination is to be performed, a specific set of preset data appropriate for that ultrasonic examination is selected manually or automatically. The selected specific set of preset data is information associated with a specific area of examination or information that indicates a specific area of examination. On the basis of this information, the image attribute is determined. In an embodiment, the image attribute corresponds to a category into which the image is classified. The server attribute corresponds to a category into which the server is classified.

In an embodiment, the ultrasonic diagnosis apparatus includes a storage unit having stored therein preset data management information for managing areas of examination for which the respective sets of preset data are used. Based on the preset data management information, the determining unit determines, as the image attribute, an area of examination for which a specific set of preset data is used. The preset data management information is, for example, information constituting a preset data management table. Information identifying an area of examination may be included in each set of preset data.

In an embodiment, the storage unit stores therein server management information for managing, as the server attribute, one or a plurality of areas of examination assigned to each server. The selecting unit identifies the transfer destination server based on the server management information. The server management information is, for example, information constituting a server management table.

In an embodiment, the storage unit stores therein image management information for managing image attributes of respective images that have been obtained and stored in an ultrasonic examination. When a transfer target image is designated from among the stored images, the selecting unit selects a transfer destination server based on the image management information and the server management information. The image management information is information included in an image management table. To each image, information for management thereof may be added.

In an embodiment, a secondary image is generated by performing processing with respect to a primary image which is an image obtained in an ultrasonic examination. When the secondary image is designated as the transfer target image, the selecting unit selects, from among a first server assigned a server attribute suitable for the image attribute of the primary image and a second server for storing secondary images, one or both of the first server and the second server as the transfer destination server. According to this configuration, the examiner's workload can be reduced also at the time of transfer of secondary images. A secondary image is an image that is derivatively generated as a result of a processing of a primary image. This processing includes measurement, analysis, three-dimensional image formation, and the like.

In an embodiment, when a training image attribute is additionally assigned to a transfer target image, the selecting unit selects, as the transfer destination server, both of a first server assigned a server attribute suitable for the image attribute of the transfer target image and a second server for storing training images. According to this configuration, transfer and accumulation of images for training use are facilitated. Designation of the training image attribute may be performed by the examiner, or may be performed automatically based on a result of image analysis.

An ultrasound image transfer method according to an embodiment includes a determining step, a selecting step, and a transfer step. In the determining step, an image attribute of an image obtained in an ultrasonic examination is determined based on operation condition setting information selected prior to the ultrasonic examination. In the selecting step, a transfer destination server assigned a server attribute suitable for the image attribute is selected from among a plurality of servers. In the transfer step, the image is transferred to the transfer destination server.

The above-described ultrasound image transfer method is implemented as a hardware function or a software function. A program for carrying out the above-described ultrasound image transfer method may be installed into an information processing device via a network or a portable recording medium. The concept of the information processing device encompasses an ultrasonic diagnosis apparatus, an ultrasonic diagnosis system, a computer, and the like. The information processing device comprises a non-transitory storage medium.

### (2) Details of Embodiments

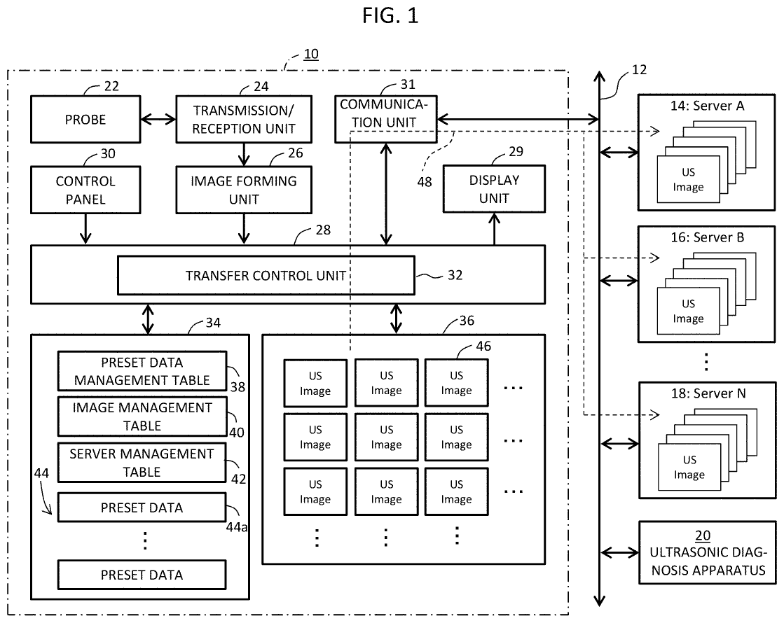

FIG. 1 shows an ultrasonic diagnosis apparatus 10 according to an embodiment. The ultrasonic diagnosis apparatus 10 is a medical apparatus that is installed in a medical institution such as a hospital and used for ultrasonic examinations.

The ultrasonic diagnosis apparatus 10 is connected to a network 12 provided within the medical institution. In the network 12, N (number) servers are connected, including a server (server A) 14, a server (server B) 16, and a server (server N) 18. Further, in the network 12, another ultrasonic diagnosis apparatus 20 is also connected. The entirety or a part of the network 12 may be the Internet.

Each server 14, 16, 18 is a file server into which images obtained by the ultrasonic diagnosis apparatuses 10, 20 are uploaded. For example, images that are designated as targets of image interpretation are downloaded from the servers 14, 16, 18 to a computer used for image interpretation. To each server 14, 16, 18, a specific area of examination is assigned as a server attribute. A plurality of specific areas of examination may be assigned to each individual server 14, 16, 18. An area of examination is a concept corresponding to a field of clinical practice, an examined body part, or the like. Typically, for each area of examination, a different ultrasonic examination is performed.

The ultrasonic diagnosis apparatus 10 will now be described in detail. A probe (ultrasound probe) 22 is a device that performs transmission of ultrasound and reception of reflected waves. In the probe 22, a transducer array composed of a plurality of transducers is provided. By means of the transducer array, an ultrasound beam is formed and electronically scanned. As electronic scan methods, an electronic linear scan method, an electronic sector scan method, and the like are known. A two-dimensional transducer array may be provided in the probe 22.

At the time of transmission, a transmission/reception unit 24 supplies a plurality of transmission signals in parallel to the transducer array, and a transmission beam is thereby formed. At the time of reception, when reflected waves from the interior of a living body are received at the transducer array, a plurality of reception signals are output in parallel from the transducer array to the transmission/reception unit 24. In the transmission/reception unit 24, the reception signals are subjected to phase alignment and summing (i.e., delay and summing), and reception beam data are thereby generated.

By performing an electronic scan of the ultrasound beam, a plurality of sets of reception beam data located serially along the electronic scan direction are obtained. Those sets of reception beam data constitute reception frame data. By repeating the electronic scan, a plurality of sets of reception frame data are generated, and those sets of reception frame data are sequentially output to an image forming unit 26.

The image forming unit 26 is a device that generates sets of display frame data from the sets of reception frame data. Specifically, the image forming unit 26 is a device that forms tomographic images which are ultrasound images, and is composed of a digital scan converter; namely, an exclusive processor. The image forming unit 26 has functions such as a coordinate conversion function, a pixel interpolation function, and a frame rate conversion function.

In the configuration example shown in drawing, the sets of display frame data are transmitted to a display unit 29 via a control unit 28. On the display unit 29, the tomographic images are displayed as a moving image. The displayed moving image is stored in response to a manipulation for storing performed by the examiner. Alternatively, a still image selected from among a plurality of still images constituting the moving image is stored in response to a manipulation for storing performed by the examiner.

The control unit 28 is composed of a processor that executes programs, and is specifically composed of a CPU. Operations of the constituent elements of the ultrasonic diagnosis apparatus 10 are controlled by the control unit 28.

Before performing an ultrasonic examination, operation conditions are set. Specifically, based on a specific set of preset data that is selected by the examiner or selected automatically, the control unit 28 sets operation conditions of the ultrasonic diagnosis apparatus. The operation conditions include the operation mode, electronic scan method, depth of diagnosis, transmission frequency, transmission power, reception gain, display conditions, display format, and the like. A set of preset data is a group of parameters that define the operation conditions.

To the control unit 28, a control panel 30 is connected, and a communication unit 31 is also connected. The communication unit 31 is connected to the network 12. The control unit 28 exchanges data with other apparatuses via the communication unit 31 and the network 12. The control panel 30 is an input device comprising switches, buttons, a keyboard, a trackball, and the like. The display unit 29 is composed of a liquid crystal display, an organic EL display, or the like.

In an embodiment, the control unit 28 has a transfer control function. This function is expressed as a transfer control unit 32 in FIG. 1. As described later, the transfer control unit 32 functions as a determining unit, a selecting unit, a transfer unit, and a transfer result managing unit.

To the control unit 28, a first storage unit 34 and a second storage unit 36 are connected. Each of the first storage unit 34 and the second storage unit 36 is composed of a semiconductor memory, a hard disk, or the like. A single storage unit may constitute the first storage unit 34 and the second storage unit 36.

The first storage unit 34 has stored therein a preset data management table 38, an image management table 40, and a server management table 42. Further, a preset data group 44 is stored in the first storage unit 34.

The preset data group 44 is composed of sets of preset data 44a that are selectively used. In accordance with an ultrasonic examination that is to be performed, a specific set of preset data 44a is selected from the preset data group 44 and used. Specifically, based on the selected specific set of preset data 44a, the operation conditions of the ultrasonic diagnosis apparatus 10 are set. With that done, the ultrasonic examination is started. For performing various ultrasonic examinations, a large number of sets of preset data 44a are provided. Each set of preset data 44a is associated with an area of examination.

The preset data management table 38 is a table for managing the sets of preset data 44a, and contains preset data management information. In an embodiment, the preset data management table 38 contains information identifying an area of examination for which each set of preset data 44a is used. The information identifying an area of examination may be included in each set of preset data 44a.

The image management table 40 is a table for managing an image attribute and the like for each stored image. The image management table 40 contains image management information. In an embodiment, as an image attribute, an area of examination is managed. Specifically, when an image (i.e., a still image or moving image) is stored during execution of an ultrasonic examination, the area of examination associated with the set of preset data being used in the current ultrasonic examination is registered as the image attribute of that image.

The server management table 42 is a table for managing a server attribute and the like for each server. The server management table 42 contains server management information. In an embodiment, as a server attribute, an area of examination is managed. In other words, one or a plurality of areas of examination are assigned in advance to each server, and the one or plurality of areas of examination are managed as the server attribute.

The second storage unit 36 stores ultrasound images 46 that were designated as targets of storage. From among these images, a specific ultrasound image or a specific group of ultrasound images are designated as the target of transfer by the examiner. Transfer target images may be designated automatically.

The ultrasound images 46 may include various images such as tomographic images, color flow mapping images, and three-dimensional images. In an embodiment, the images are transferred using the DICOM (Digital Imaging and Communications in Medicine) protocol, which is an international standard protocol, or the like.

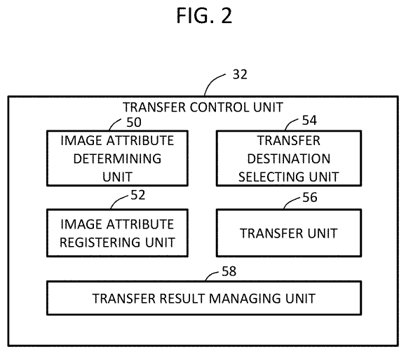

In FIG. 2, the functions of the transfer control unit 32 are represented by blocks. The transfer control unit 32 comprises an image attribute determining unit 50, an image attribute registering unit 52, a transfer destination selecting unit 54, a transfer unit (transfer executing unit) 56, and a transfer result managing unit 58. The transfer control unit 32 also has the functions to create and modify the preset data management table 38, the image management table 40, and the server management table 42.

When an image designated by the examiner is to be stored, the image attribute determining unit 50 determines the image attribute of that image. Specifically, the image attribute determining unit 50 refers to the preset data management table, and identifies the area of examination associated with the currently-used set of preset data. Subsequently, the image attribute determining unit 50 determines the identified area of examination as the image attribute of the image obtained in the current ultrasonic examination. The image attribute registering unit 52 registers the determined image attribute in the image management table 40. In an embodiment, since the image attribute is determined and registered at the time of storing of images, the image attribute of a transfer target image can be identified instantly at the time of image transfer. However, the image attribute may alternatively be determined at the time of image transfer.

When a transfer target image is designated, the transfer destination selecting unit 54 refers to the image management table, and identifies the image attribute of the transfer target image. Subsequently, the transfer destination selecting unit 54 refers to the server management table so as to identify a server assigned a server attribute suitable for the image attribute of the transfer target image, and selects that server as a transfer destination server. Specifically, the transfer destination selecting unit 54 selects, as the transfer destination server, a server that satisfies the condition of selection that the area of examination is the same. The transfer unit 56 transfers the transfer target image to the transfer destination server.

When the server attribute suitable for the image attribute is assigned to a plurality of servers, the transfer target image is transferred to those servers in parallel. The examiner is only required to designate or select the transfer target image, and does not need to select the transfer destination server for the transfer target image. The selection of the transfer destination server is performed automatically by the transfer control unit 32. Accordingly, the examiner's workload at the time of image transfer can be reduced significantly, and each transfer target image can be correctly transferred to its appropriate transfer destination server.

The transfer target image may be designated automatically. Further, before executing a transfer, information regarding the transfer destination server may be displayed on the display unit. In that case, the image may be actually transferred when a confirming input is received from the examiner.

The transfer result managing unit 58 serves to manage, for each image transfer, the result of the transfer; namely, a success or failure of the transfer. Specifically, transfer successes and failures are managed for each transfer destination server.

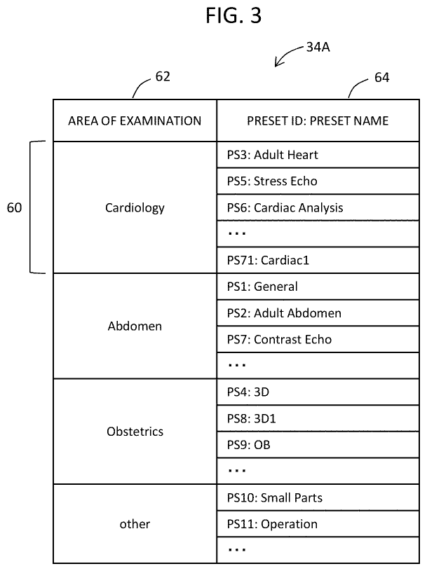

FIG. 3 shows a first example of the preset data management table. The preset data management table 34A shown contains records 60 corresponding to respective areas of examination. Each record 60 contains information 62 identifying an area of examination, and information 64 identifying one or a plurality of sets of preset data associated with that area of examination. In the example shown, each set of preset data is identified by a preset ID and a preset name. Here, the areas of examination are, for example, the cardiac area, the abdominal area, the obstetrics area, and other areas.

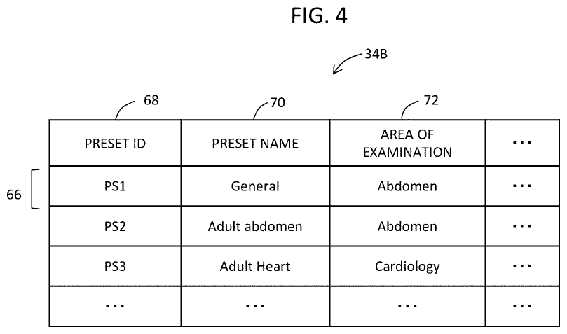

FIG. 4 shows a second example of the preset data management table. The preset data management table 34B contains records 66 corresponding to respective sets of preset data. Each record 66 contains information such as preset ID, preset name, and area of examination. The area of examination is information that identifies a field of clinical practice or a living body part for which the set of preset data is used. Information identifying an area of examination may be included in each set of preset data.

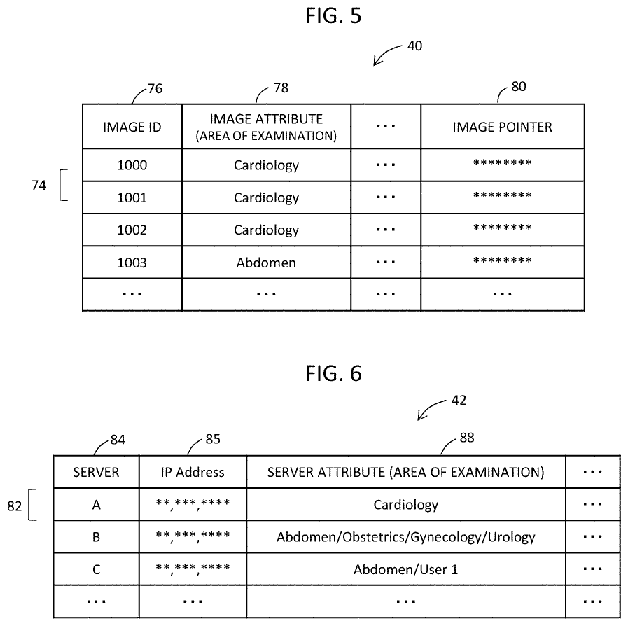

FIG. 5 shows an example of the image management table 40. The image management table 40 contains records 74 corresponding to respective stored images. Each record 74 contains an image ID 76, an image attribute 78, and the like, and further includes an image pointer 80. The image pointer 80 is data identifying the location of the image. As the image attribute 78, the area of examination determined by the image attribute determining unit is registered.

FIG. 6 shows an example of the server management table 42. The server management table 42 contains records 82 corresponding to respective servers. Each record 82 contains a server 84, an IP address 85, a server attribute 88, and the like. The server attribute 88 is the area of examination assigned to the server.

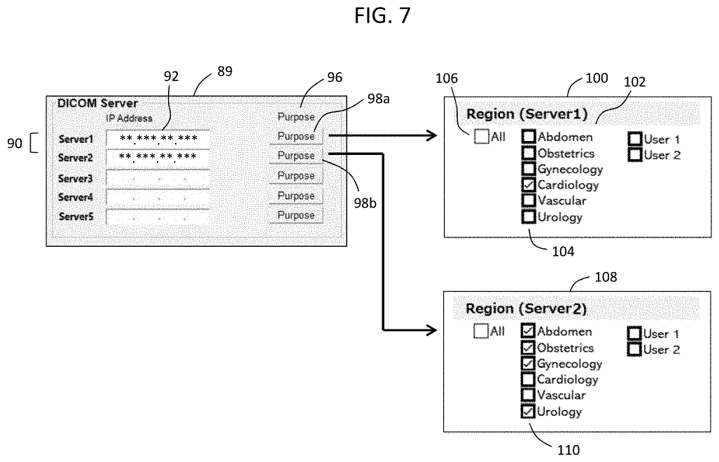

FIG. 7 illustrates a method of designating server attributes. First, a settings screen 89 is displayed. The settings screen 89 contains settings entries 90 corresponding to respective servers. Using each settings entry 90, an IP address 92 is set. By manipulating buttons 98a, 98b included in the respective settings entries 90, windows 100, 108 are displayed. Use of the windows 100, 108, server attributes can be designated.

The window 100 contains a list 102 of areas of examination and checkboxes 104. By manipulating the checkboxes 104, server attributes are designated. When it is desired to assign all areas of examination, a special checkbox 106 is manipulated. The window 108 has a configuration identical to that of the window 100. As indicated by reference numeral 110, a plurality of areas of examination are selected simultaneously.

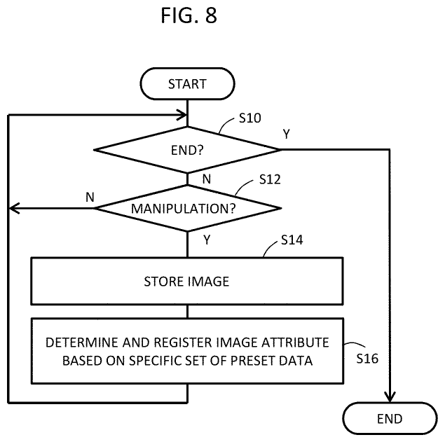

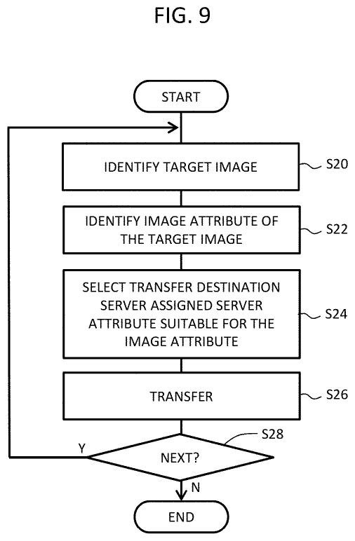

Next, operations and controls performed by the transfer control unit will now be described by reference to FIGS. 8 and 9. FIG. 8 illustrates an example operation at the time of image attribute registration, and FIG. 9 illustrates an example operation at the time of transfer.

In FIG. 8, in S10, whether or not to end the present process is determined. In S12, a determination is made as to whether or not a manipulation for storing has been performed. When a manipulation for storing has been performed, in S14, a store target image is stored into the second storage unit of the ultrasonic diagnosis apparatus. In S16, based on a specific set of preset data selected in advance, an image attribute is determined, and this image attribute is registered in the image management table. In determining the image attribute, reference is made to the preset data management table. The foregoing process is repeatedly executed.

In FIG. 9, in S20, a transfer target image is identified based on a designation by the examiner. In S22, the image attribute of the transfer target image is identified. At that time, reference is made to the image management table. In S24, a server assigned a server attribute suitable for the identified image attribute is selected as a transfer destination server. At that time, the server management table is referred to. In S26, the transfer target image is transferred to the transfer destination server. In S28, presence or absence of a subsequent transfer target image is determined. When a subsequent transfer target image is present, the steps from S20 are executed again.

As described above, in the ultrasonic diagnosis apparatus according to an embodiment, a transfer destination server suitable for the transfer target image is selected, and the transfer target image is transferred to that transfer destination server. Accordingly, the examiner is not required to designate or select a transfer destination at the time of image transfer. As a result, the examiner's workload can be reduced, and incorrect selection of a transfer destination server can be avoided.

During an ultrasonic examination, at a point when an image is stored, that image may be automatically transferred. After completion of an ultrasonic examination, at each time a transfer target image is designated from among a stored group of images, the designated image may be transferred. After completion of an ultrasonic examination, a stored group of images may be automatically sorted and transferred. On each day of examination, after completion of the last ultrasonic examination, the entirety or a part of a group of images stored on that day of examination may be automatically sorted and transferred. When a transfer of a certain image results in a failure, at a point after elapse of a predetermined time therefrom, that image may be automatically retransferred. A screen for setting or selecting a transfer schedule may be provided to the examiner.

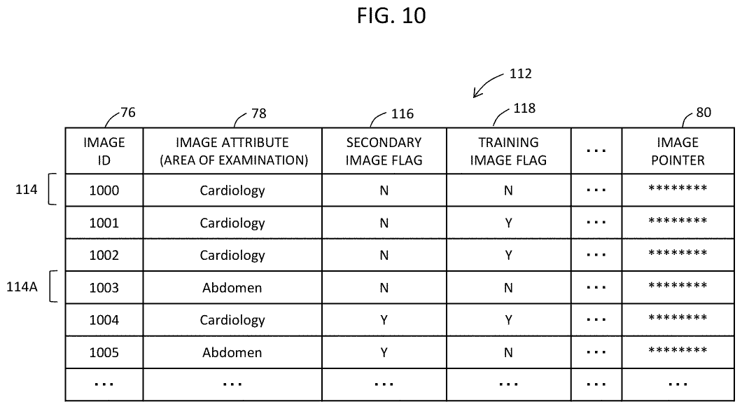

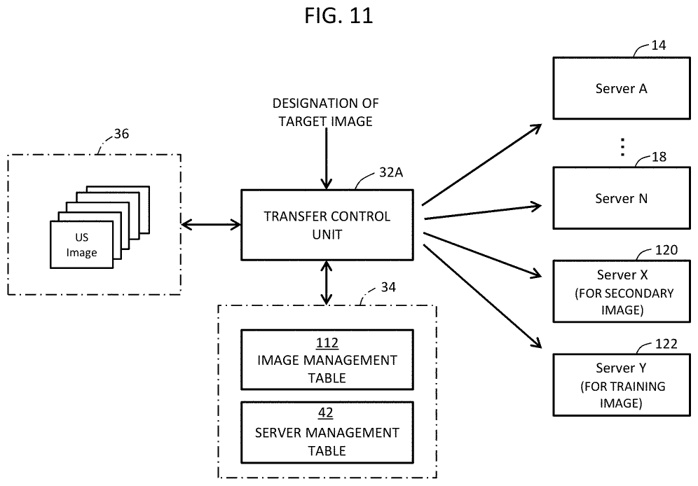

FIG. 10 shows another example of the image management table. FIG. 11 schematically illustrates a transfer control according to a variant example, which is performed using the image management table shown in FIG. 10.

In FIG. 10, the image management table 112 contains records 114 corresponding to respective stored images. Each record 114 contains an image ID 76, an image attribute 78, a secondary image flag 116, a training image flag 118, and an image pointer 80. As the image attribute 78, an area of examination is registered. The secondary image flag 116 is information identifying whether or not the image is a secondary image. The training image flag 118 is information identifying whether or not the image is a training image. The secondary image flag 116 may also be referred to as an image category flag. The training image flag 118 may also be referred to as a purpose designation flag.

When a stored image (i.e., a primary image) is reproduced and subjected to a measurement or analysis, the measurement result or analysis result is reflected in the image. The image having the result reflected therein is a secondary image. A secondary image contains information (such as graphics and numerical values) that are not included in a primary image. A secondary image is an image derived from a primary image, and is, for example, an image including a result of measurement or analysis of the primary image. A three-dimensional image generated from a plurality of primary images may serve as a secondary image. A secondary image is stored in response to a manipulation for storing performed by the examiner or stored automatically.

Based on the secondary image flag 116, whether or not the stored image is a secondary image (i.e., whether or not the image has the secondary image attribute) can be identified. When a stored image is designated as a training image by the examiner, a training image attribute is assigned to that image. Based on the training image flag 118, whether or not the image is a training image can be identified.

In FIG. 11, the first storage unit 34 and the second storage unit 36 are connected to the transfer control unit 32A. As transfer destinations, a server 14, . . . a server 18, a secondary image storing server (server X) 120, and a training image storing server (server Y) 122 are provided. When the transfer control unit 32A recognizes a designation of a transfer target image, based on that designation, the transfer control unit 32A identifies the transfer target image in the second storage unit 36, and reads out that transfer target image. Further, the transfer control unit 32A refers to the image management table 12 in the first storage unit 34, and identifies the image attribute of the transfer target image. At the same time, the transfer control unit 32A refers to the secondary image flag and the training image flag assigned to the transfer target image, and determines whether or not the transfer target image is a secondary image, and also determines whether or not the transfer target image is a training image.

Subsequently, the transfer control unit 32A refers to the server management table 42, and selects, as a transfer destination server, a server assigned the server attribute suitable for the image attribute of the transfer target image. Apart from this selection, when the transfer target image is a secondary image, the secondary image storing server 120 is selected as a transfer destination server. Further, when the transfer target image is a training image, the training image storing server 122 is selected as a transfer destination server. After that, the transfer control unit 32A transfers the transfer target image to the selected one or plurality of transfer destination servers.

Accordingly, when a secondary image is designated as a transfer target image, that image is transferred to the secondary image storing server 120. Further, when a training image is designated as a transfer target image, that image is transferred to the training image storing server 122. When a primary image has already been transferred and a secondary image corresponding to that primary image is designated as a transfer target image, that secondary image may be transferred to only the secondary image storing server 120.

In an embodiment, the servers to be designated as transfer destination servers are information processing devices that are physically independent from each other. However, each of those servers may alternatively be a logical server. A computer used for image interpretation or a specific folder located therein may be designated as a transfer destination server. A recording medium (including a USB memory) may be designated as a transfer destination.

In the above-described embodiment, instead of the area of examination, a different attribute may be designated as the image attribute and the server attribute. Examples of the different attribute include the operation mode, image category, name of disease (or symptom), and the like. While the preset data management table, the image management table, and the server management table are provided in the above-described embodiment, the preset data management information, the image management information, and the server management information may be managed by another method.

1. An ultrasonic diagnosis apparatus, comprising:
a determining unit configured to determine, based on operation condition setting information selected prior to an ultrasonic examination, an image attribute of an image obtained in the ultrasonic examination;
a selecting unit configured to select, from among a plurality of servers, a transfer destination server assigned a server attribute suitable for the image attribute; and
a transfer unit configured to transfer the image to the transfer destination server.

2. The ultrasonic diagnosis apparatus according to claim 1, wherein
a plurality of sets of preset data are provided,
each of the sets of preset data includes parameters that define operation conditions of the ultrasonic diagnosis apparatus,
the operation condition setting information is a specific one of the sets of preset data selected prior to the ultrasonic examination, and
the determining unit determines the image attribute based on the specific one of the sets of preset data.

3. The ultrasonic diagnosis apparatus according to claim 2, further comprising
a storage unit having stored therein preset data management information for managing areas of examination for which the respective sets of preset data are used, wherein
based on the preset data management information, the determining unit determines, as the image attribute, an area of examination for which the specific one of the sets of preset data is used.

4. The ultrasonic diagnosis apparatus according to claim 3, wherein
the storage unit stores therein server management information for managing, as the server attribute, one or a plurality of areas of examination assigned to each server, and
the selecting unit identifies the transfer destination server based on the server management information.

5. The ultrasonic diagnosis apparatus according to claim 4, wherein
the storage unit stores therein image management information for managing image attributes of respective images that have been obtained and stored in the ultrasonic examination, and
when a transfer target image is designated from among the stored images, the selecting unit selects the transfer destination server based on the image management information and the server management information.

6. The ultrasonic diagnosis apparatus according to claim 1, wherein
a secondary image is generated by performing a processing with respect to a primary image which is an image obtained in the ultrasonic examination, and
when the secondary image is designated as a transfer target image, the selecting unit selects, from among a first server assigned a server attribute suitable for an image attribute of the primary image and a second server for storing secondary images, one or both of the first server and the second server.

7. The ultrasonic diagnosis apparatus according to claim 1, wherein
when a training image attribute is additionally assigned to a transfer target image, the selecting unit selects, as the transfer destination server, both of a first server assigned a server attribute suitable for an image attribute of the transfer target image and a second server for storing training images.

8. An ultrasound image transfer method, comprising:
determining, based on operation condition setting information selected prior to an ultrasonic examination, an image attribute of an image obtained in the ultrasonic examination;
selecting, from among a plurality of servers, a transfer destination server assigned a server attribute suitable for the image attribute; and
transferring the image to the transfer destination server.

9. A program configured to be executed by an information processing device, the program having:
a function to determine, based on operation condition setting information selected prior to an ultrasonic examination, an image attribute of an image obtained in the ultrasonic examination;
a function to select, from among a plurality of servers, a transfer destination server assigned a server attribute suitable for the image attribute; and
a function to transfer the image to the transfer destination server.
